(19) United States
(12) Patent Application Publication
(10) Pub. No.: US 2026/0195892 A1
(43) Pub. Date: Jul. 9, 2026

(54) GENERATING COMBINED DIAGNOSTIC IMAGING AND PATHOLOGY IMAGES

(71) Applicant: KONINKLIJKE PHILIPS N.V., EINDHOVEN (NL)

(72) Inventors: TOBIAS WISSEL, LÜBECK (DE); CHRISTIAN BUERGER, HAMBURG (DE); RENAT KOZODAYEV, EINDHOVEN (NL); GEORGII KOLOKOLNIKOV, HAMBURG (DE)

(21) Appl. No.: 19/136,312

(22) PCT Filed: Dec. 6, 2023
(86) PCT No.: PCT/EP2023/084428

§ 371 Date: Jun. 6, 2025

(30) Foreign Application Priority Data
Dec. 9, 2022 (WO) PCT/CN2022/137930
Jan. 17, 2023 (EP) 23151957.0

(51) Int. Cl. G06T 7/00; G16H 10/60; G16H 50/20

(52) U.S. Cl. CPC G06T 7/0012; G16H 10/60; G16H 50/20; G06T 2207/10088; G06T 2207/20081; G06T 2207/20084; G06T 2207/20121; G06T 2207/30024

(57) ABSTRACT

A computer-implemented image generation method (100) for generating combined diagnostic imaging and pathology images is described. The method (100) comprises: acquiring with a first imaging modality pathological image data (110) of a subject; acquiring with a second imaging modality diagnostic image data (120) of the subject; mapping (140) the pathological image data to the diagnostic image data; and applying an image generation algorithm (150) in order to generate a combined diagnostic and pathology image based on the mapping of the data.

### FIELD OF THE INVENTION

The invention relates to generating combined diagnostic imaging and pathology images, which may be used in the field of clinical decision support systems.

### BACKGROUND OF THE INVENTION

Diagnostic imaging like Computer tomography (CT), Ultrasonography (US), Magnetic Resonance Imaging (MR) has long been the standard of diagnostics for many diseases. However, for such diseases as cancer, diagnostic imaging alone might be insufficient. This is the case since performing the imaging scanning alone might run the risk of missing cancer lesions for multitude of reasons (Missed Lesions at Abdominal Oncologic CT: Lessons Learned from Quality Assurance, Bettina Siewert et. al., https://pubs.rsna.org/doi/10.1148/rg.283075188). Diagnostic imaging exams always runs a risk of missing on cancer lesions for many reasons such as human mistakes, small cancer cells that cannot be detected by imaging modality, incorrectly configured diagnostic imaging device, etc.

In many cases if there is a suspicion of cancer occurring in a patient, even if it is not confirmed by the diagnostic imaging modality, an additional pathological examination is being performed. Pathological examination and pathological imaging data provides a rich source of additional information for diagnostics of a patient. Pathological examination typically involves using a pathology system acquiring means (e.g., a biopsy device), sample imaging means (e.g., a microscope) and monitoring means (e.g., a workstation with software) for analyzing the images. However, analyzing both diagnostic imaging data and pathological imaging is a challenging task in clinical practice. Therefore, improvements in the field are needed.

US2020364864A1 describes a system and method for generating normative imaging data for medical imaging processing using a Deep Learning algorithm. The document focuses on applying anomalous data of one image to anomalous data of another image, which is described as a normative image.

### SUMMARY OF THE INVENTION

There is a need to improve the diagnostic procedures for complex diseases such as oncology, cardiac diseases, autoimmune diseases, diabetes, and others. One of the complexities stems from the fact that in these diseases both diagnostic imaging and pathological data needs to be combined for correct diagnosis. For instance, in oncology diseases, the diagnostic imaging studies are time-efficient, but are error-prone due to different human and machine specifics and errors. Furthermore, in diagnosis of cancer, the diagnostic imaging modalities like US, MR, CT cannot provide such information as morphology of the disease. That is why in many cases, a second pathological study is ordered for supply of the imaging data with additional pathological information.

However, ordering and management of the diagnostic and pathological data creates multiple clinical problems associated with this workflow. For instance, it is difficult to navigate where the puncture should be done and where the biopsy should be performed. It is also challenging for the physicians to analyze the data after the diagnostic and pathological exams have been performed. Radiologists are trained in review of imaging studies, such as Ultrasound (US), Computed Tomography (CT), or Magnetic Resonance Imaging (MRI) studies, but are not usually trained in review of pathological examinations. To make it even more challenging, some radiologists are only trained in review of Magnetic Resonance (MR) images and are usually not too experienced in US and/or CT images, which creates additional challenges. Pathologists on the other hand are trained in review of pathological images, but are not trained in review of US, CT, MRI images. This also creates multitude of challenges during performing the procedure.

At the same time, for efficiently diagnosing the disease, information from both diagnostic imaging and pathological imaging modalities needs to be analyzed. One way to alleviate this, is to participate in Tumor Board meetings. However, even in Tumor Board meetings, in many cases it is difficult to review the case since each person is trained in his/her own field, and not sufficiently trained in other fields.

Hence, there is a need for improvements in the field of diagnostics of such complex diseases as oncology. There is a need to combine diagnostic imaging information/data and pathological imaging information/data. Specifically, there is a need to combine the information from diagnostic imaging modalities, such as US, MRI and CT, with the information from the pathological imaging modalities, such as stained tissue analysis done through a Digital Pathology scanner. Specifically, there is a need to generate images combining both diagnostic and pathological image information. This might be for preserving lesion boundaries considering imaging modality information and generating pathological image information. Furthermore, there is a need for generating high-quality diagnostic images comprising high-resolution pathological image information with sufficient image resolution.

It is an object of the present invention to solve these challenges in clinical practice. Specifically, it is an object of the present invention to provide a device and corresponding method that allows to combine information from diagnostic images coming from a diagnostic imaging modality with information from pathological images coming from a pathological imaging modality. Furthermore, it is an object of the present invention to provide a method and corresponding device for mapping diagnostic images with pathological images, and generating a combined image containing both diagnostic and pathological information. It is a further object of the present invention to present a Machine Learning (ML) algorithm that is used for generation of such images.

According to examples in accordance with an aspect of the invention, a computer-implemented image generation method for generating combined diagnostic images coming from a diagnostic imaging modality (e.g., US, MRI, CT) and pathology images coming from a pathology imaging modality is disclosed. The method comprises the steps of:
acquiring with a first imaging modality pathological image data of a subject;
acquiring with a second imaging modality diagnostic image data of the subject;
   mapping the pathological image data to the diagnostic image data; and
applying an image generation algorithm in order to generate a combined diagnostic imaging and pathology image based on the mapping of the data. In some embodiments, the image generation algorithm (150) is a machine learning algorithm refined based on the mapping (140) information, and wherein the image generation algorithm (150) is further configured for determining cellular information from the pathological image data of biopsied regions of a subject, and extrapolating this information to non-biopsied regions for determining cellular information of non-biopsied regions. It is to be understood that mapping of the data comprises mapping of diagnostic to pathological data or vice-versa.

The method may further include visualizing the combined pathological image. The method may further include registering the pathological image data and diagnostic image data from the two modalities. In an exemplary embodiment, this may be done before the mapping step.

In an embodiment, the first pathological imaging modality is of pathological or histopathological type. Within the context of the present invention “pathological imaging modality” and “pathological images” are an umbrella terms combining different images that contain some morphological information from a patient. These images can include, but are not limited to pathological images, histopathological images, and other images containing cell morphology information. As a representative example, the first diagnostic imaging modality could be of microscope slide scanner type (also known as digital pathology or digital histology scanner). In general, the first pathological imaging modality could include any scanner that is used for analysis of pathological images, such as histopathology scanner, histology scanner, pathology scanner, digital pathology scanner, microscope, or any other scanner that could be used for analysis and representation of the tissue information and analysis of pathology slides.

The second diagnostic imaging modality may be an ultrasound or High Frequency Ultrasound (HFUS) modality type. However, other modalities can be used within the context of the present invention, such as, CT, High Intensity Focused Ultrasound (HIFU), MRI, Positron Emission Tomography (PET), Single Photon Emission Computed Tomography (SPECT), PET/MR, PET/CT, X-ray imaging, SPECT/CT, SPECT/MR, and others.

Within the context of the present invention, referring to first pathological imaging modality and second diagnostic imaging modality may refer also to the images generated from such modalities, and both can be used interchangeably. Other variations in naming the “first pathological imaging modality” and “second diagnostic imaging modality” may exist. For instance, first and second imaging modalities, pathological modality and imaging modality, etc. But essentially these are synonyms, which will be apparent for the skilled person.

Registering of images from the first and second imaging modalities may involve image registering/transforming sets of data for each of the first and second imaging modalities into one coordinate system. Mapping of images may involve e.g., translating one set of images to another set of images (further details would be given below). After or during the registration/mapping, an image generation algorithm might be applied. The image generation algorithm is configured to generate a combined image containing registered histopathological image data and registered diagnostic image data. Any image generation algorithms, like image fusion algorithms, image-to-image translation algorithm, synthesizing images and others can be used. Visualization of images may involve a process of converting/rendering pixels/voxels from the mapped images into 2D/3D graphical representation. Consequently, an image containing both information from a first pathological imaging modality (e.g., histopathology scanner) and a second diagnostic imaging modality (e.g., US or HFUS) can be obtained.

In some embodiments, the image generation method and associated device is configured to generate synthetic images containing both diagnostic image and pathological image information. It is to be understood that “generating the synthetic images” or “generating a combined image” are used as examples, and other ways of combining images can be used. For instance, image fusion through asynchronous image combination, image fusion through synchronous image combination, and other methods known within the field. It is to be understood that the combined image could be a synthetic, fused or any other image type being able to contain information from both the first and second imaging modalities.

Within the context of the present invention, the combined image could be referred to as “enhanced histopathological image”. However, this is purely for clarity reasons and does not limit the invention in any possible way. It is to be understood that the data acquired by first and second imaging modalities could be acquired both synchronously, i.e., in parallel at the same point in time, and asynchronously, i.e., at different points in time. It is to be understood that the steps of registering and planning can also be applied synchronously and asynchronously.

In some embodiments, a Neural Network might be used for generation of the combined image containing both diagnostic imaging and pathology imaging data. In an embodiment, the Neural Network algorithm is of a Generative Adversarial Network (GAN) algorithm, a diffusion model algorithm, a convolutional neural network and/or transformer-based neural network. Other types of machine learning or neural network type of algorithms can be used.

In some embodiments, an image generation method, wherein the image generation algorithm is a Generative Adversarial Network (GAN) or a multi-conditional generative neural network (MC-GAN) type algorithm is disclosed. In some embodiments, the image generation method is disclosed, wherein the algorithm is configured to: encode the data with an encoder, apply a multi-layer perceptron, calculate the mean and variance, apply a product-of-expert algorithm, calculate the mean and variance, apply a multi-layer perceptron, create a latent representation, and decode and generate the combined image. Other variations of this algorithm are possible, for instance, omitting some of the steps, depending on the goal at hand.

In some embodiments, an image generation algorithm is disclosed, wherein the pathological image data and the diagnostic image data are further mapped with a diagnostic image with a biopsy needle, wherein a further biopsy region extraction and applicational of segmentation mask is performed.

In some embodiments, a confidence map is generated for determining the confidence on extrapolated information of non-biopsied regions.

In some embodiments, based on the mapping, at least one of or combination of: biopsy information, biopsy positions and/or radiological image at biopsied positions is determined. Optionally, the information is fed to the image generation algorithm for improving functioning of the algorithm. Optionally, the image generation algorithm can generated a whole-organ histology image.

In some embodiments, a biomarker extraction module can be further used. The biomarker extraction module can be used for instance for calculating cellular information values directly from the diagnostic image data. Other uses of biomarker extraction module can be envisaged, for instance, for calculating biomarker information from the biopsy information and/or from radiological image information. Optionally, the biomarker extraction module predicts histological information for each of the locations in the radiological image data other than locations corresponding to the pathological image data of biopsied regions based on the mapping. Optionally, the biomarker extraction module is based on a pointwise machine learning algorithm. In some embodiments, the biomarker extraction module is used for calculating cellular information values from the pathological image data of biopsied regions and extrapolated to non-biopsied regions.

It is to be understood that the cellular information may comprise any one of: Gleason score, cell growth progression, cell growth markers, lesion shape, cell diffusion. This is a non-exhaustive list, and other values may be envisioned within the context of the present invention. Optionally, based on the determined cellular information, a biomarker vector is calculated. It is to be understood that the biomarker vector is a scalar vector representative of any one of molecular, histologic, radiographic, or physiologic characteristics of the molecules. In some cases, multiple biomarker vectors can be aggregated together to calculate the characteristics values over a certain region.

Optionally, biomarker vectors can be used as input for the image generation algorithm, wherein the image generation algorithm is configured for determining N biopsy locations in Region of Interest, ROIs, for which biomarker vectors have been measured.

In some embodiments, the method further comprises generating at least one whole-organ histology image comprising of diagnostic image data, and pathological image data of biopsied and extrapolated non-biopsied regions. It is to be understood that the histology image is a representative example, and generally it is a fused image comprising of information from both pathological image data and diagnostic image data, whereas the advantage of such image is that a whole organ, whole region and/or whole image data is generated reflective of both radiological and/or histological information in a single image with a particular advantage that non-biopsied regions are provided with potential biopsy (extrapolated) information based on the available information.

In some embodiments, mage intensities are calculated for the diagnostic image data, wherein image intensities are used for generating image patches based on the diagnostic image data. Preferably, the image patches are generated for MR image types. Optionally, the pathological image data of biopsied regions is used for calculating image patches comprising the biopsied regions, wherein the image generation algorithm determines the cellular information, preferably in a form of bicellular biomarkers, for center points of at least some image patches. Optionally, a patch-wise prediction for image patches of non-biopsied regions is determined, and a confidence scores for generated image patches are calculated.

In some embodiments, the diagnostic image data is MR image data, and on the MR image data, MR intensities are calculated. Optionally, the MR intensities could be used as a proxy for calculating of the cellular information, i.e., if an intensity is within a certain range among other associated information, this could be an indication of a lesion, and then the algorithm will reflect this in the prediction.

In some embodiments, the determined cellular information of the current patient is fed into a refinement module, wherein the refinement module is used for a calculating cellular information for a new patient based on the cellular information of the current patient. In this way, the cellular information for new patients could be potentially determined as a proxy from the current patient by comparison of the diagnostic image data and/or pathology image data of a new patient with the old patient. This could be a beneficial embodiment in case of e.g., cancer screenings. Optionally, the refinement module is configured to adopt the model parameters of the image generation algorithm based on an optimization objective similar to the training of patient-specific biopsy information, and/or wherein the refinement module is configured to calculate model parameters based on the patient-specific biopsy information.

In some embodiments, the image generation algorithm is configured to learn and synthesize the pathological representations covering the whole or part of tissue in the diagnostic image data.

In some embodiments, the diagnostic image data is MR image data, and wherein the MR image data is registered with live US image data from an ultrasound machine, wherein at least one of the following model parameters are determined: MR image intensities, cellular markers, biopsy locations in the MR image, and wherein based on these parameters, a confidence registration map is determined, the confidence registration map being reflective of registration certainty of MR image data with live US image data. In some embodiments, a further image-to-image (I2I) algorithm is applied. The I2I algorithm is configured to map the registered histopathological image to the diagnostic image data. Furthermore, the image generation algorithm, such as the GAN algorithm, might be used for image-to-image translations. In some embodiments, by using both a GAN and I2I algorithm, the precision of the mapping can be increased. The image generation algorithm may be configured to learn and synthesize the pathological representations covering the whole or part of tissue in the diagnostic image data. The mapping may include mapping contours of a part identified as pathological in the pathological image data to the diagnostic image data, or vice-versa.

In other words, in some instances the image generation algorithm might be configured to identify and synthesize image parts, like a contour, on the image generation algorithm or the whole image. Combination of both generation of parts and general images can be used. By “image parts” or “biopsy region extraction” region it is understood that a specific part of an image is identified and synthesized on the diagnostic image data. This would allow to specifically focus on regions of interest. One non-limiting example to apply this algorithm is to map the boundaries and/or pixels from the pathological image data to diagnostic image data. For instance, mapping a tumor from a pathological image data to a diagnostic image data.

In some embodiments, an image segmentation algorithm is applied, which is configured to segment the tissues from the histopathologic image data to diagnostic image data. The image generation algorithm may be further configured to determine the similarity of the pathological image data and the diagnostic image data. The segmented image data may be used as input for the image generation algorithm, and the image generation algorithm may be further configured to determine the similarity of histopathological and diagnostic image data. This may be based on the information from the segmented image. In some embodiments, the image generation method may be used, wherein the segmented image data is being used as input for the image generation algorithm, and the image generation algorithm is further configured to determine the similarity of histopathological and diagnostic image data.

In some embodiments, the method further includes mapping and/or translating regular shapes to irregular shapes from the pathological image data and the diagnostic image data. The image generation algorithm may be further configured to map and/or translate regular shapes in the first set of image data to irregular set of image data in a second set of image data. For instance, the pathological image data might contain irregular cancer shapes, which might be mapped towards regular organ shapes in the diagnostic images to enhance the image. One practical way may to determine the similarity is to use standard deviations in both the diagnostic image data and pathological image data and compare the two. This would increase the precision of the algorithm. In some embodiments, an image generation method is described, wherein the method further includes mapping and/or translating regular shapes to irregular shapes from the pathological image data and the diagnostic image data.

The diagnostic imaging modality can be selected from any or combination of: Ultrasonography (US) image, High Frequency Ultrasound (HFUS) image, Magnetic Resonance (MRI) image, Computer Tomography (CT) image. In an embodiment, HFUS or US modality is used.

In some embodiments, a mapping of histopathological image to diagnostic images is achieved in the range of 20:1, preferably 10:1, even more preferably 1:1. In an embodiment, it is possible to achieve a 1:1 mapping between the diagnostic imaging data and the histopathological imaging data. The algorithm can be further configured to be trained on the histopathological data and/or the diagnostic imaging data to produce better mapping of the algorithms based on the data deployed. In some embodiments, a region of interest of a nodule is determined. The determination could be automatic by the image generation algorithm or manual based on the collected user input.

A system for generating synthetic images from at least two imaging modalities is disclosed comprising at least two imaging modalities. The first imaging modality may be selected from the following modalities: a microscope scanner, a histopathology scanner, digital pathology scanner. The second imaging modality may be selected from the following modalities: Ultrasound (US), High-Frequency Ultrasound (HFUS), Computer Tomography (CT), Magnetic Resonance Imaging (MRI). In an embodiment, the combination includes digital pathology scanner and HFUS/US modalities.

In some embodiments, a cloud communication media, such as a cloud storage, can be used with the described system to transmit the data. Cloud communication media is not limiting in any way and could include any of: cloud storage, data lakes, data ponds, and other cloud communication and storage media. The cloud communication media/cloud storage represents the node in the communication link that links hospital nodes like slide scanners, enterprise software, diagnostic imaging modalities, PACS system or other nodes. The cloud communication media/cloud storage represents the cloud software servers with the related programs that would be able to obtain and store the data from the e.g., hospitals.

A computer program element for controlling an apparatus described which when executed by a processor is disclosed. The computer program element is configured to carry out the method and/or to deploy the training algorithm as described herein.

One of the technical advantages of certain embodiments of the current invention is that the combination of the pathological and diagnostic imaging information would allow to produce images with enhanced diagnostic value containing both diagnostic imaging and pathological data.

Another technical advantage may reside in that the image translation, mapping and generation algorithm(-s) allows for more specific mapping/registration of images from different imaging modalities. For instance, by using an image translation algorithm, it is possible to train the algorithm to have an accurate mapping of the diagnostic images to pathological images.

Another advantage may reside in that similarity of the histopathological and diagnostic image information can be determined, which results in more accurate mapping of images.

Another advantage may reside in that images from different image types and/or from different images slices (with e.g., varying slice thicknesses) can be generated.

A further advantage may reside in that by using the current algorithm, an accurate mapping of irregular shapes to regular shapes can be achieved.

Another advantage resides in generation of a synthetic image from modalities of different slice thicknesses.

Another advantage may reside in improving the clinical workflow as there is no need for sharing the images between departments, but a combined image can be generated. In some instances, this image is generated in real-time.

Another advantage may reside in generating cellular information for regions that have not been biopsied.

Another advantage may reside in displaying cellular information in a region of a diagnostic image.

Other advantages can be envisaged to the person of the ordinary skill in the art within the context of the present invention.

These and other aspects of the invention will be apparent from and elucidated with reference to the embodiment(s) described hereinafter.

### BRIEF DESCRIPTION OF THE DRAWINGS

FIG. 1 schematically depicts an image generation algorithm according to an embodiment of the present invention.

FIG. 2 describes the method and system of an embodiment of the present invention linking it towards the clinical perspective.

FIG. 3 describes a non-limiting example of an image segmentation algorithm that can be applied to any embodiment of the present invention.

FIG. 4 describes an embodiment, wherein the segmentation mask is used for further improving the generation of images from the first and second diagnostic image modalities.

FIG. 5 describes an MC-GAN algorithm example according to the present invention.

FIG. 6 describes an example of training the MC-GAN algorithm according to the present invention.

FIG. 7 described in-detail the extrapolation algorithm according to the present invention.

### DETAILED DESCRIPTION OF EMBODIMENTS

The invention will be described with reference to the figures. The person of ordinary skill in the art would understand that further embodiments may be possible within the context of the present invention.

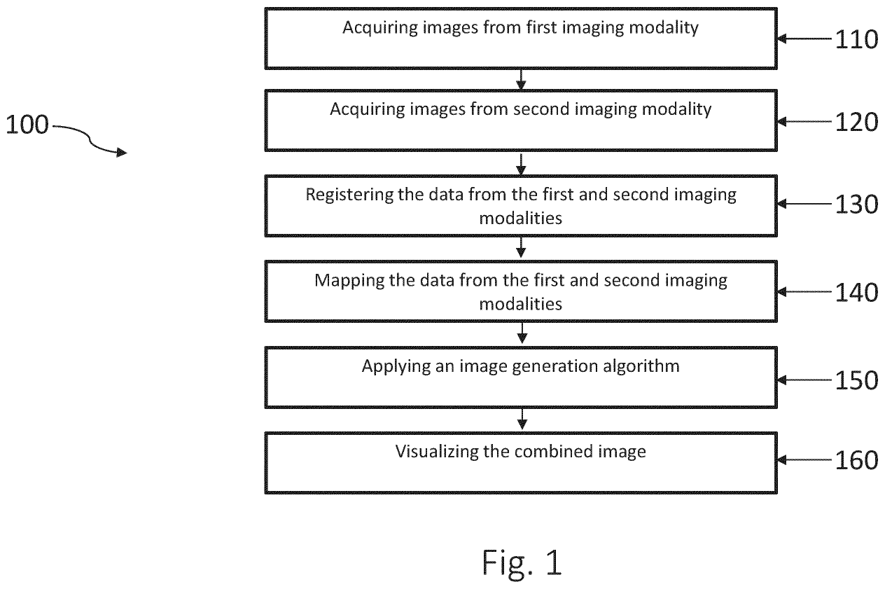

The detailed description and specific examples, while indicating exemplary embodiments of the apparatus, systems, and methods, are intended for purposes of illustration only and are not intended to limit the scope of the invention. These and other features, aspects, and advantages of the apparatus, systems and methods of the present invention will become apparent from the following description, appended claims, and accompanying drawings. The FIGS. 1-6 are merely schematic and are not drawn to scale. It should also be understood that the same reference numerals (e.g., 100, 200, 300) are used throughout the Figures to indicate the same or similar parts.

Within the field of diagnostics of human disease, a multitude of different devices are used to provide the diagnostics of the patient diseases. Within clinical diagnostics, there are 3 most common device types for performance of diagnostics: 1. clinical/laboratory diagnostic equipment, such as electrocardiographs hematology analyzers; 2. Radiology diagnostic equipment, such as Ultrasound (US), Magnetic Resonance Imaging (MRI) and other imaging equipment; and 3. Tissue diagnostic equipment, such as pathology scanners or tissue processing systems.

Different device types are providing different information points, which is used for diagnosing the patient. For instance, diagnostic imaging equipment like Ultrasound (US) is sufficiently accurate in imaging the general organ structure but is not sufficiently accurate to image cancer cells due to inherent resolution properties. This is because sound waves echo's differently from fluid-filled cysts and solid masses. Hence, an US device can reveal tumors that may be cancerous, but also have sufficient chances of missing these lesions due to associated technological complexities. MRI, on the other hand, is sufficiently accurate in analyzing the soft tissues of the patient but is prone to artifacts/variations in the devices, as well as is one of the costliest procedures that cannot be used on a routine basis.

Pathology scanners are fairly accurate in analyzing the morphological characteristics of a tissues but are highly dependent on which part of tissue is being taken for further analysis (e.g., if the affected cell is captured or not), as well as provide slides of significant size, that are challenging to manage within clinical practice. As a non-limiting example, assuming a biopsy slide output of 1,600/day and an average of 2 GB/slide, 1 PB/year should be calculated if all scans are to be archived and used by the clinical institution. Combining this with the diagnostic information is a challenging task.

In many cases, for such complex diseases as cancer, infectious diseases, chronic diseases, inflammatory conditions, thyroid lesions, diseases involving sterile body cavities (e.g., peritoneal, pleural, and cerebrospinal), there is a need to use more than one modality. In many cases radiology diagnostic equipment (diagnostic imaging modalities such as the secondary diagnostic imaging modalities) and tissue diagnostic equipment (pathological imaging modalities such as primary pathological imaging modalities) are complementing each other and providing complementary information points for clinicians to consider. However, in many cases there are challenges in using both of these equipment types in clinical practice.

For instance, the referring physician might refer the patient to have a radiological examination followed by a pathological examination. The referring physician (e.g., Radiation Oncologist) will receive the two pieces of information (diagnostic radiological images and pathological images), yet this will leave many challenges on interpretation of the diseases, as the referring physician might not be an expert in radiology or pathology studies to be able to analyze the studies. Even for experts in the field like Radiologists and Pathologists with the current complexity of technologies and diseases, there exists a challenge in analyzing the results of the studies. This is especially the case if e.g., a Pathologist needs to analyze radiological images, which is frequent in the current clinical environment. To give an example in relation to cancer, the physicians are gathering for Tumor Board meetings to discuss the patient's condition and determine a course of treatment. Yet, this is still quite a complicated procedure taking a lot of time, prone to errors. Furthermore, Tumor Board meetings are quite expensive and cannot be used routinely for e.g., screening purposes.

However, combination of information/data from two different imaging modalities of two different imaging modality types is very important for such complex diseases as cancer. The information contained in the pathology scanners is fundamental to understand the morphology and pathology of the cancer treatment. Usually, a diagnostic exam is performed to understand the patient's anatomy and if regions of cancer are present, which will subsequently help in performing the pathological examination. The diagnostic imaging scan is done through a CT, MR or US modality, wherein a Radiologist is then evaluating the results to diagnose cancer cells. If presence of cancer cells has been determined, then a follow-up pathology scan is being performed. In some cases, this is done even if no cancer cells have been spotted on the imaging modality, but there is a suspicion of a cancer in the patient. The pathological examination can be done through taking biopsy samples of a tissue of a patient, staining the tissue samples through e.g., Hematoxylin and Eosin (H&E) staining, and subsequent visualizing the samples with a pathology scanner or associated workstation.

One of the major challenges for the Pathologist and Interventional Radiologist is to understand the anatomy of the patient correctly for correctly performing the biopsy, as well as consequently correctly analyzing the pathology sample considered patient anatomy. It would be apparent for the skilled practitioner, that to improve the diagnostic quality, the e.g., Pathologist or Interventional Radiologist performing the biopsy, needs to consult the diagnostic imaging data to correctly determine the patient's anatomy. This may prove quite cumbersome, time-consuming, and prone to many mistakes in the interpretation. To perform the pathology examinations, the Pathologist needs to well define the boundary of the lesion or organ with lesions. This creates further mistakes of human and technical nature. From the human side the diagnostic imaging scans might be misinterpreted and the place of the cancer incorrectly identified. From the technical perspective diagnostic image information and pathological image information has different information structure and the combination of information is prone to many mistakes. Yet, considering the valuable clinical information that is provided by both modalities there is a pressing clinical need to combine the information for more efficient diagnosis of complex diseases like cancer.

Even though there is the desire to limit the number of invasive biopsies to the absolute minimum required, they will remain the gold standard for tissue characterization. Although technical advances in other medical fields have made it possible to predict tissue markers from more macroscopic radiological images (such as Virtual Histology (VH) analyzing plaque on intravascular ultrasound images), it is unlikely that predictions of models that generalize their way of data processing over a limited corpus of training data are going to replace these definite insights for clinical reasoning. Furthermore, limited or unknown algorithmic confidence and the ignorance with respect to patient-specific histological information constitute major shortcomings of current approaches that try to translate whole-organ ROI radiological information to cellular biomarkers. Moreover, for prostate cancer even such an algorithm is not available. On the other hand, merging of radiological and histological information from biopsies for enhanced tumor prognosis prediction exists, but operates only very localized and restricted to the biopsy locations.

One of the realizations of the inventors of the present invention is that there is a need to combine information from two different imaging modalities of different modality types in order to assist the physicians in diagnosis of the tumor. As a non-limiting example, the first imaging modality could be of Radiology diagnostic equipment type, such as Ultrasound (US) or Magnetic Resonance Imaging (MRI) equipment; and the second imaging modality could be of tissue diagnostic equipment type, such as a digital pathology scanner. By combining the information from different imaging modality types, the quality of diagnosis of a patient and the clinical workflow can be improved, with reduced costs to the clinical institution. Currently, there are no approaches used in clinical practice combining information from two different imaging modalities of two different modality types preserving the information in both the modalities. The current approaches are limited to combination of two imaging modalities from the same type. For instance, for in radiation therapy treatment, a multi-modality simulation of radiological images (e.g., simulation of CT to MR data) is used.

Furthermore, the inventors have realized that an machine learning algorithm, such as a generative AI algorithm, such a GAN or MC-GAN could be used for generating a an image combining pathology and radiology information. Especially the knowledge on the existing cancerous regions and associated information could be used as a proxy for generating pathology information predictions on other organs based on e.g., a comparison of existing image intensities of cancerous tissues with other regions. It is to be understood that it is a non-limiting example and other approaches described previously and/or that will be described subsequently are possible within the context of the present invention. In some instances, algorithm (re-)training taking into account images of different patients may be possible. In some cases, conditioning of patient-specific information may be possible. In some cases, given the cellular information, the algorithm would be able to model extrapolates, impute or otherwise determine the remaining histological indicators for the desired region in the radiological image, which has not necessarily been biopsied. In some instances, this might be beneficial for avoiding more invasive tissue samples where the model is already confident, and providing a macroscopic overview (e.g., by providing phenotype information) about the tissue marker spread over the whole organ or specific ROIs of interest.

In some embodiments, a method of training an image generation algorithm is disclosed, wherein the algorithm is trained based on the diagnostic image data, or based on both the diagnostic image data and the pathologic image data, and wherein the training is done based on a Generative Adversarial Network (GAN) algorithm, a diffusion model algorithm, a convolutional neural network and/or transformer-based neural network. This list is non-limiting. One potential embodiment might comprise a conditioned generative model, which could be potentially especially useful in a patch to biomarker implementation. However, for patch-to-biomarker implementation, unconditioned e.g. convolutional or transformer architectures, where the biopsy information enters as a target for overfitting only during refinement, but not during actual extrapolation might be more preferred.

The insights of the inventors are schematically described in FIG. 1. FIG. 1 schematically depicts the image generation algorithm according to the present invention. In the first aspect of the present invention, a computer-implemented image generation method, or just an image-generation method 100, for generating combined diagnostic and pathology images is described. The method comprises the steps of:
acquiring with a first imaging modality pathological image data 110 of a subject;
acquiring with a second imaging modality diagnostic image data 120 of the subject;
   mapping 140 the pathological image data to the diagnostic image data;
applying an image generation algorithm 150 in order to generate at least one combined diagnostic and pathology image based on the mapping of the data. In some embodiments, the image generation algorithm (150) is a machine learning algorithm trained based on the mapping (140) information, and wherein the image generation algorithm (150) is further configured for determining cellular information from the pathological image data of biopsied regions of a subject, and extrapolating this information to non-biopsied regions for determining cellular information of non-biopsied regions.

In a practical non-limiting clinical example, this might be done through execution of the following procedure: after acquiring an MR image, regions of interest for biopsy probing are delineated. By registering (e.g., by using by UroNav of Philips or other ultrasound equipment) the live US images to this MR during the image-guides biopsy, samples are taken from all desired locations and histologically, e.g., pathologically, morpologically or otherwise, analysed and cellular information is determined. The cellular information could be in for of (bio-)markers that are extracted. In some cases, this could be done based on determination of e.g. Gleason score prevalent for determination of prostate cancers. In some embodiments, all three informative components, namely, MR image intensities, cellular markers and the biopsy locations in the MR image are passed to a machine learning algorithm, preferably of generative AI type. Optionally, the algorithm could be used for refinement based on these 3 patient-specific support feature sets, before it extrapolates the cellular information, such as marker information, onto the rest of the organs and/or ROI of the diagnostic image, such as MR image. Optionally it can provide a confidence map about its extrapolation output.

In some embodiments, the at least one combined image may be visualized for representation. Other ways in presenting the image exist. In some embodiments, there is an optional registration step 130 done before the mapping step 140. The goal of the registration step is to increase the precision of the mapping algorithm and to reduce the number of errors. Variations of the approach in respect to FIG. 1 might occur depending on the embodiment. Further details describing the approach of the current invention will be given below.

Combination of data from two different imaging modalities of different modality types, such as diagnostic and pathological imaging modalities, proves to be quite difficult to perform in order to maintain the advantageous information contained in both imaging modality types. Fundamental issue in combination of data from two different imaging modality types is the different slice thicknesses of the modalities. For instance, the slice thickness of in-vivo radiology images is around 1.5 mm for MRI and 0.8 mm for CT. The determination of slice thickness for ultrasonography images is a more complicated task and depends on many factors like the depth of the imaging. However, the slice thickness of an Ultrasound machine still varies in the range from 0.1 to 2 mm for most applications. On the other hand, the slice thickness of images from a pathology scanner is around 5 μm. Comparing CT images with pathological images, would yield a difference of around 1000:1. Hence, the images of the diagnostic modalities are not exactly overlapping or matching the appearance of gross pathology or histopathology due to such stark differences. Consequently, in combination of images, many valuable diagnostic information can be lost in the process.

The inventors of the current invention have realized that there might be instances, where there is a need to combine the information from two different imaging modalities in order to have a better diagnosis and come up with an algorithm for doing so. In an embodiment, the inventors have devised an algorithm that acquires data from a first pathological imaging modality 110 (e.g., digital pathology scanner), acquires data from a second diagnostic imaging modality 120 of a radiological type (e.g., US, CT, MR). Then the image generation method 100 performs a mapping 140 of the pathological data towards the diagnostic data. The mapping may include registering 130 the data from the pathological data to the diagnostic data, or vice versa from the diagnostic data to the pathological data. Finally, an image generation algorithm 150 is applied to the mapped data sets in order to generate a combined diagnostic imaging and pathology image based on the mapping of the data. As a final step, acquired data may be visualized 160. This method allows to generate images with combined diagnostic and pathological data. It is to be understood that preferably the algorithm generates cellular information from the regions of biopsies and extrapolates this information to non-biopsied regions, thereby obtaining e.g., a whole organ image comprising both radiological information and cellular information. In some embodiments, a degree of confidence can be displayed. Preferably, the algorithm for generating of images is a machine learning algorithm.

It is to be understood that in order to correctly combine the data, first an image registration may need to be performed. Image registration within the context of the present invention, can be defined as the process of aligning two or more images, wherein the images can be of the same imaging modality (e.g., US to US) or different modalities (e.g., US to pathological images), or both. Image registration can also be called image fusion, matching or warping within the context of the present invention.

The goal for applying an image registration method is to find the optimal transformation that best aligns the structures of interest in the input images of first imaging modality and second imaging modality types. Accurate registration of images is an important step for correctly preserving the information of the two different images and correctly aligning the images. The data from the diagnostic imaging modality can be in a DICOM (Digital Imaging and Communications in Medicine) format since it relates to diagnostic images, such as CT, US, MR, or others. Any of the algorithms used in the field in relation to DICOM image registration can be used. As a non-limiting example, the algorithms used for image registration of diagnostic images could be of: intensity-based image registration type, spatial registration methods, time-series registration methods, extrinsic registration methods, intrinsic registration methods, landmark-based registration methods, segmentation-based registration methods, voxel property-based registration methods. In some embodiments registration is synonymous to segmentation. For more information in relation to image registration techniques, reference is made to the document Image registration methods: a survey, by Barbara Zitová (https://www.sciencedirect.com/science/article/pii/S0262885603001379), incorporated by reference.

At least some of these methods can be applied also for the pathological images. Due to the nature of the pathological images, registration methods for pathological images may be: intensity-based registration methods (e.g., normalized mutual information), feature-based registration methods (e.g., scalar-invariant feature transform), feature/intensity-based registration methods (e.g., Register Virtual Stack Slices, RVSS), or segmentation based methods (e.g., ASSAR). The reasoning for using these image registration methods is that the pathological images are usually stained, and the described methods allow to achieve a better registration for pathological images in view of the landmark points. Both elastic and non-elastic registration methods can be used.

In some cases, multiple machine learning algorithms could be used, and the algorithm output could be stacked together, to form a stacked images. Such approaches could be particularly useful in cancer diagnosis due to complexity of cancer treatment. One way to do it is described in the document Stacking Machine Learning Algorithms for Biomarker-Based Preoperative Diagnosis of a Pelvic Mass by Reid Shaw et. al., (available at https://www.ncbi.nlm.nih.gov/pmc/articles/PMC8909341/).

The image registration techniques used in this invention could be intensity-based and/or feature based. Feature-based techniques according to this invention may involve spatially transforming the source/moving image(-s) to align with the target image. The reference frame in the target image is stationary, while the other datasets are transformed to match to the target. Intensity-based methods according to this invention may involve comparing intensity patterns in images via correlation metrics, while feature-based methods find correspondence between image features such as points, lines, and contours. Intensity-based methods register entire images or sub-images like image regions. If sub-images are registered, centers of corresponding sub images are treated as corresponding feature points. Feature-based methods establish a correspondence between several distinct points in an image. Both linear and elastic transformations may be used according to the present invention.

In some embodiments, the image generation algorithm is further configured to be refined based on the cellular information from one patient, and wherein the image generation algorithm is further optimized to analyze the data of the one patient before the algorithm extrapolates the information to non-biopsied regions. In this case, the cellular information is used as an optimization target criterion, wherein the refinement is done to reach the target criterion, and wherein upon reaching the criterion, the image generation algorithm only then will perform the needed steps.

In some embodiments, cellular biomarkers are extracted from the pathological data, and the cellular markers are fed into the machine learning algorithm as an optimization target. In this embodiment, the model does not act upon the pathological or cellular information directly, but acts upon the extracted cellular markers.

In some embodiments, an extraction module is used for the extraction of cellular information, and wherein the extracted cellular information is fed to the machine learning algorithm, wherein the extraction module has fixed parameters used for extraction of cellular information. Fixed parameters mean that the parameters are fixed during the process, and the cellular information is not used for refinement of the algorithm, and only for extraction. In this case, the image generation module only is used for determining the cellular information of interest based on the extracted cellular information by the extraction module.

In some embodiments, the diagnostic images 120 are directly aligned and registered to pathological images 110. In some embodiments, first the diagnostic images are registered to each other (e.g., an US image is registered to another US image), the pathological images 110 are registered to other pathological images 110, following by a registration from the pathological images 110 to diagnostic images 120. While one registration method may be used for both diagnostic and pathological images, it is to be understood that different registration methods may be used depending on the goal. Any combination of the methods described in the previous paragraph can be used. For instance, to align diagnostic images to other diagnostic images 120 spatial registration methods can be used, whereas for registering the pathological images 110 to each other, and then registering the pathological images 110 to diagnostic images 120, an intensity-based registration methods can be used.

In some embodiments, the images can be pre-processed before the registration of the images is done. Applying a pre-processing algorithm can increase the alignment results. In some embodiments, the system is displaying the result of the registration of the images to the user. In this way, the user has the possibility of changing the results of the registration to achieve a better alignment of images. In some embodiments, annotation can be done by the registered pair of images (diagnostic to diagnostic, pathology to pathology, or diagnostic to pathology). In some embodiments, an image similarity metric can be used for comparing and correctly registering the diagnostic and pathology imaging modalities. The similarity metric can take the two image intensity values for a certain image and returning a scalar value that describes how similar the images are. Furthermore, an optimizer can be used, wherein optimizer defines the methodology for minimizing or maximizing the similarity metric.

After performing the optional registration 130 of images, or during the process of registration, image mapping 140 is being performed. In mapping one image onto another image, a mechanism is used to match and find the corresponding spatial regions which have the same meaning between the source and the matching image. By doing so, the information in the images can be preserved, which will subsequently be displayed in the image containing both radiological and pathological data. Image mapping is preferred to be used, since the underlying diagnostic and pathological image data have different structures. Mapping 140 within the context of the present invention may involve mapping between spatial regions in the source, and matching images in a database. The mapped regions have similar semantics, which improve preservation of data from the images. Image-based data integration is useful for integrating data from various information modalities. In some embodiments, mapping of images is also followed by image segmentation algorithms. Any known image segmentation algorithms known in the field, can be used, such as: (1) manual delineation methods, (2) low-level segmentation methods, and/or (3) model-based segmentation methods. The Applicant incorporates by reference the information from the document Medical Image Segmentation Techniques: An Overview, by E. A. Zanaty (https://www.researchgate.net/publication/294682473_Medical_Image_Segmentation_Techniqu es_An_Overview). AI methods for segmentation can also be applied within the context of the present invention. For instance, contour-based segmentation methods, voxel-based segmentation methods, registration-based segmentation methods, fully convolutional networks (FCN's), U-net's, dilated convolutional networks, and others.

According to the present invention, mapping 140 of one image onto another image involves a mechanism to match and find the corresponding spatial regions with the same meaning between the source and the matching image. Image-based data integration is useful for integrating data of various information structures. In some embodiments, fiducial points in diagnostic and pathological images 110, 120 are being determined, and image segmentation of fiducial points and the corresponding anatomical regions is being performed. This helps to further improve translation of points from the diagnostic images towards the pathological images.

The image generation algorithm 150 is applied based on the registered and mapped images. Image generation within the context of the present invention has the same meaning as within the field of medical imaging: it is a process of synthesizing new medical images. The process of generating medical images 150 will be described in-detail below.

Within the context of the present invention, the first pathological imaging modality 110 can refer to any modality suitable for analyzing tissue information, and providing information on the morphology of the tissues, organs or fluids. This can refer to gross, microscopic, immunologic, genetic and molecular imaging modalities to determine the presence of disease. In an embodiment, the pathological imaging modality is of digital pathology type. Further, the second diagnostic imaging modality 120 can refer to any diagnostic imaging modality. Ultrasound (US) or High-Frequency Ultrasound (HFUS) modalities may be used. However, other imaging modalities are possible. Non-limiting examples include MRI, CT, PET/MR, PET/CT, SPECT, X-ray, CBCT, angiography, fluoroscopy and other imaging modalities.

In some embodiments, the image generation algorithm is a Generative Adversarial Network (GAN) or a multi-conditional generative neural network (MC-GAN) type algorithm. In some embodiments, the image generation method is configured to: encode 351 the data with an encoder, apply 352 a multi-layer perceptron, calculate 353 the mean and variance, apply 354 a product-of-expert algorithm, calculate 355 the mean and variance, apply 356 a multi-layer perceptron, create 357 a latent representation, and decode 358 and generate 359 the combined image. Other variations of this algorithm are possible, for instance, omitting some of the steps depending on the goal at hand.

In some embodiments, the pathological image data 110 and the diagnostic image data 120 are further mapped with a diagnostic image with a biopsy needle, wherein a further biopsy region extraction and applicational of a segmentation mask is performed.

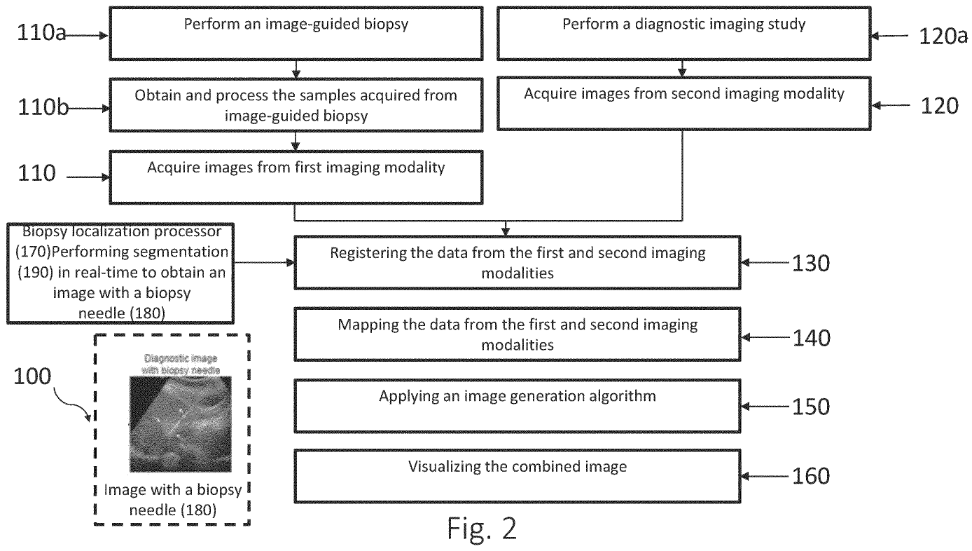

FIG. 2 describes an embodiment of the method and system of an embodiment of the present invention linking it towards the clinical perspective.

In getting the required data, the user would usually perform an image-guided biopsy 110a. In performing 110a the image-guided biopsy, the user would obtain 110b tissue samples, which then need to be processed. By “processing” within the context of the present invention, the standard handling of tissue samples used for pathological analysis is used. For instance, the biopsied tissue is put into small containers (cassettes), processing of samples for fixating the tissue to the cassette is done (e.g., in hot paraffin wax), cut into thin slices with a microtome, specimens put on glass slides, and dipped into a series of stains or dyes to change the color of the tissue (by using e.g., H&E staining). The obtained images are then stored in a database, which are further acquired by the device 100 for further analysis. Performing 120a a diagnostic imaging study within the context of the present embodiment follows conventional procedure of obtaining diagnostic images. For instance, the patient might come in an examination room, the physician is analyzing the patient with an ultrasound probe, the images are stored on the US device, and later may be transmitted to a database. It is to be understood, that the pathological images and diagnostic US images are given as non-limiting representative example, and other combination of images (e.g., cytology and MR images) are possible, or combination of modalities (e.g., US and MR modality with histopathology scanners) is possible.

The diagnostic images 120 are acquired from e.g., the database, or the US device, for further analysis. When the system 100 acquires the images 110 from the first pathological image modality and the images 120 from the second diagnostic image modality, the system 100 registers 130 the data from the first and second imaging modalities, maps 140 the data from the first and the second imaging modality, applies 150 an image generation algorithm, and visualizes 160 the resulting image.

In some embodiments, the images 110 from the first imaging modality and the images 120 from the second imaging modality are acquired simultaneously. The biopsy needle navigation can be obtained by e.g., a guided fusion biopsy system, like the Uronav system of Philips. The Uronav system fuses pre-biopsy MR images of the prostate with ultrasound-guided biopsy images in real time. This allows to improve the delineation of the prostate and suspicious lesions, as well as clear visualization of the biopsy needle path.

In order to correctly perform 110a the image-guided biopsy procedure, a biopsy localization processor and corresponding method of biopsy localization 170 can be used. The biopsy region localization processor 170 may be based on a Convolutional Neural Network (CNN), such as a lightweight convolutional neural network. The CNN performs semantic segmentation in real-time, and shows the result on a diagnostic display, such as the display of an ultrasound machine. The convolutional neural network first down-samples the input diagnostic images from the second diagnostic imaging modality with trained convolution-based down-sampling layers. There are two options when the down-sampling is performed: 1. The down-sampled data can be used for fusing the images of second diagnostic imaging modality with the biopsy needle 180. 2. A further global feature extraction is performed in order to reduce the dimensionality of the data, whereas afterwards an image fusion as the one described in option 1 is performed. By using the second option, it would be possible to apply classification of pixels on a hierarchical level.

Furthermore, real-time image segmentation can be applied. The real-time segmentation may be of a semantic image segmentation type. The real-time image segmentation is configured focus on 3 sections of the image in the segmentation process: the biopsy needle, the one or more lesions, the image background. However, other focus points are possible. For instance, the algorithm can focus on one or more fiducials, which could be identified by the user or an algorithm. A diagnostic image with a biopsy needle is generated as an output 190.

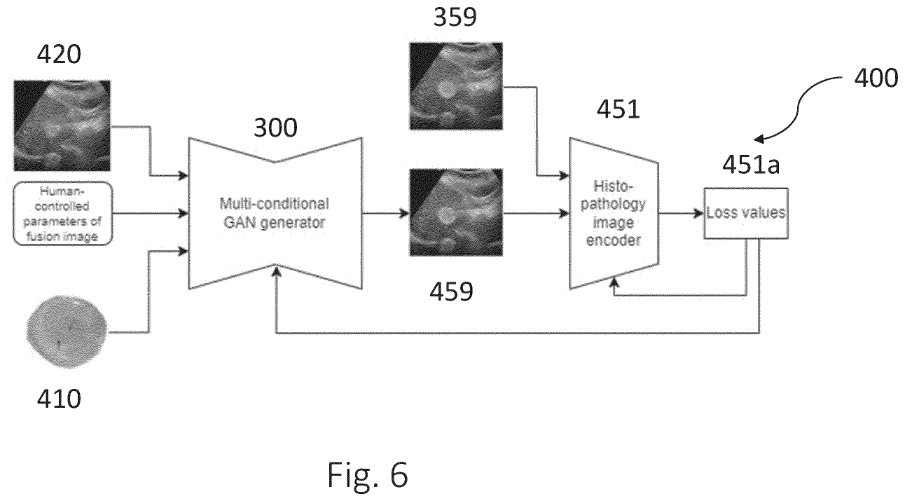

In order to further increase the precision of the algorithm, the CNN network can be trained on one or more data sets. In an embodiment, training of the CNN network may be performed on the dataset, that contains diagnostic images from the second diagnostic imaging modality, wherein biopsy needle and associated data (e.g., fiducial associated with the biopsy needle) as inputs for the model, and wherein based on the inputs and the CNN algorithm, the segmentation is performed. As an example, the segmentation of the biopsy needle can be performed. Training of the data can be conducted both on a supervised manner and non-supervised manner. The training may be done in a supervised manner. A non-limiting example of a training method is given in relation to embodiment of FIG. 6. As a representative example, the output may include a diagnostic image with biopsy image and respective segmentation showing biopsy localization.

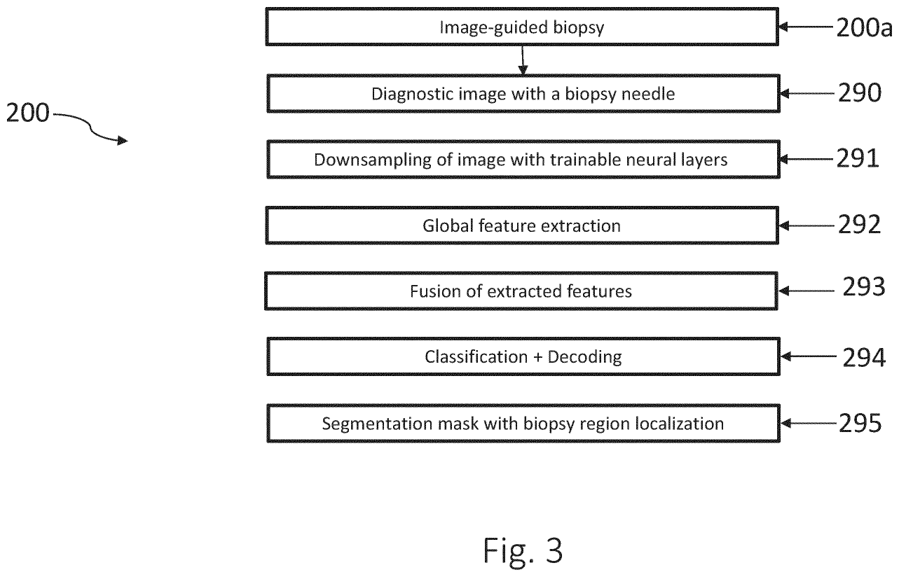

FIG. 3 describes a non-limiting example of an image segmentation algorithm that can be applied to any embodiment of the present invention. FIG. 3 describes that an image-guided biopsy 200a is performed, wherein by performing the steps described previously, a diagnostic image with a biopsy needle is obtained 290. After performing the down-sampling 291 of the image with trainable neural network layers in a form of a fully connected layers or convolutional layers, the CNN network performs global feature extraction 292. Subsequently, down-sampled image representations before (291) and after feature extraction (292) are fused 293. After the fusion of images has been performed, a classifier and decoder can be applied 294 in order to classify the images, e.g., to classify the images on the diagnostic images. After the classification is performed, a segmentation mask with a biopsy region localization is generated 295. In some embodiments, the generated localization mask could be used for combination of images.

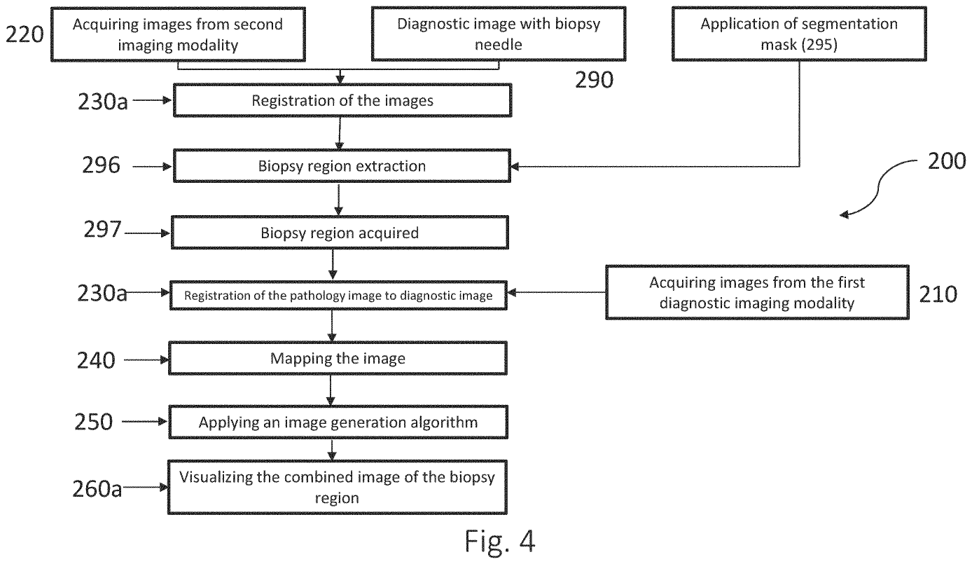

FIG. 4 describes an embodiment, wherein the segmentation mask is used for further improving the generation of images from the first and second diagnostic image modalities 210, 220. FIG. 4 describes acquiring first two sets of data, namely acquiring images 220 from the second diagnostic imaging modality (e.g., US), and acquiring diagnostic images 290 with biopsy needle. As a non-limiting example, the diagnostic images 290 with a biopsy needle could be preoperative, intra-operative, or combination of the two image types. When the images 220, 290 are obtained, they are registered 230a to each other by any of the methods described herein. The notion “230a” instead of “230” is used to indicate that in this case the image pair 220, 290 is registered. For the original pair of images 210, 220, the notation “230” is used. When the images are registered, a biopsy extraction 296 may be performed. Extraction means identification of a region of interest, i.e., the region of interest, where the biopsy will be likely/is performed. Biopsy extraction 296 may mean any means performed in the art to emphasize the extracted region. For instance, the biopsy extraction region may be delineated, may be segmented, mapped out, emphasized (e.g., highlighted), delimited, outlined, or identified in any other way that is suitable in the field of image registration and image visualization.

The goal of the biopsy extraction 296 is to identify the region of interest, where the biopsy is performed. The biopsy extraction 296 may also mean identification of other points of interest in relation to the biopsy extraction, e.g., identification of lesions or fiducials. After or during the biopsy extraction 296, the device and the associated computer-implemented algorithm of the present invention can apply a segmentation mask 295 to the biopsy region, i.e., the region of interest associated with the biopsy procedure in the diagnostic image. Segmentation mask 295 could be of the type described in relation to FIG. 3. However, other segmentation mask approaches are possible within the context of the present invention. In an embodiment, the segmentation mask 295 is of type that is suitable for instance segmentation. A non-limiting example of such a mask could be R-CNN. Application of the segmentation mask 295 could be done after the biopsy region extraction step 296 is performed, or while the step 296 is being performed. In some embodiments, application of the segmentation mask 295 is optional. When the biopsy region extraction 296 and the application of the segmentation mask 295 is performed, the region of the biopsy is acquired 297.

By acquiring biopsy region 297 within the context of the present invention, a sequence of steps is meant for making the image available for further registration, e.g., storing the image on a (Random-Access Memory) RAM/storage device, like hard-disk drive/transmitting to the cloud, and/or converting the image to a specified data format. When the biopsy region is acquired 297, the system may acquire the images from the first diagnostic imaging modality 210. The images from the first imaging modality may contain e.g., pathological images. In some embodiments, images from the first diagnostic imaging modality may be acquired from the previous examinations, e.g., from the previous biopsy studies. By incorporating the images from the previous biopsy studies, there is a possibility of improving the clinical practice, in e.g., making a more precise determination of the affected regions with e.g., cancer, so a more accurate biopsy can be taken. This is important taking into account the non-homogeneity of the cancer cells, and hence such a structure would allow to more precisely taking the biopsy samples from multiple section of the affected region. This could be useful for both screening and diagnosis purposes. In some embodiments, the pathological data can be received from the pathological studies done in real-time. Upon receiving the data from the first imaging modality 210, and the registered 230a data from the second imaging modality 220 and the diagnostic image with biopsy needle, a further registration 230 of the registered data 230a and the data from the first imaging modality 210 is performed. The registration could be of any type previously described. Upon registration 230, a mapping may be performed 240, an application of an image generation algorithm 250 and visualizing the combined image 260a. Notion “260a” is used to specify that this image also contains data from the diagnostic image with a biopsy needle 290, however notion “260a” is otherwise similar to the notion “260”.

The following example explains the clinical perspective of having such a method more in-detail. Once a biopsy sample is extracted from the patient, it is examined with a digital pathology scanner or other histopathology equipment, which results in a pathology image (i.e., image from the first imaging modality 210). Previously acquired diagnostic image from the first imaging modality 220, diagnostic image with biopsy needle 290, and the acquired image from the first imaging modality 210 are being registered in step 230a. Registering the images from the first imaging modality 210 (e.g., histopathologic image) to the registered images from the second imaging modality 220 and the diagnostic image with a biopsy needle 290 is a challenging task. This is mainly to the following challenges:
1. Preparation of a biological sample for pathological/histopathological examination can introduce artifacts. Non-limiting examples of artifacts may include deformations, shrinkages, tissue ripping. By correctly registering the images, at least some of these artifacts can at least partly be avoided. For instance, deformation artifacts and shrinkage artifacts can be alleviated by correctly registering the images, while other artifacts, such as tissue ripping artifacts, can be addressed by correctly applying the image generation algorithm 260 (in other words correctly synthesizing the images).
2. Lesion appearance in diagnostic images, e.g., the ones obtained from the second diagnostic imaging modality 220, drastically differ from the lesion observed in a pathological image, e.g., the images acquired from the first pathological imaging modality 210. There are many reasons for that, e.g., different slice thicknesses of modalities, staining involved in pathological images, different image formats, etc. In order to address this, it would be useful to have additional information, such as the extracted biopsy region 296 to alleviate this challenge.

It is to be understood that the registered segmentation mask may be used to extract biopsy region from the diagnostic image from the second diagnostic imaging modality 220. It is to be further understood, that the output of the image registration 230a may include pairwise registered diagnostic 220 and pathological 210 images, wherein the pathological images 210 may contain artifacts that image registration could not remove (e.g., tissue ripping artifacts). Image registration 230a may be performed by optimizing affine and deformable transforms using a registration based on a multi-resolution pyramid with three layers. The image registration may be done either on the device of 200, or any other suitable device, such as the workstation of a physician containing the images.

Further, examples of image generation algorithms 160, 260 that can be used in previous embodiments 100, 200 are described. The image generation algorithm 160, 260 could be of any type of image generation algorithm: image fusion algorithm, existing image transformation, image regeneration, or any other methods of combining information from one image to the second image. In an embodiment, the image generation algorithm 160, 260 is of image fusion type. The image fusion algorithm may be based on Machine Learning, such as a GAN algorithm. A non-limiting example is given below.

In a non-limiting example, the image generation algorithm can be based on multi-conditional generative neural network (MC-GAN). The technical details of such algorithm are incorporated by reference from Huang, Xun, et al. “Multimodal conditional image synthesis with product-of-experts gans.” arXiv preprint arXiv: 2112.05130 (2021). In the current invention, the image generation method can be based on generative adversarial neural network, which acquires the diagnostic image from the second imaging modality 120, 220 diagnostic image, the diagnostic pathology image from the first imaging modality 110, 210. It is to be understood that in some embodiments, also the diagnostic image with the biopsy needle 290 or the registered image 230a can be used as input for the algorithm. In some embodiments, the user can change the parameters of the images and input additional parameters for the MC-GAN algorithm, which is called “human-controlled parameters” within the context of the present invention. This means that the MC-GAN can be applied to any of the steps, e.g., in relation to mapping, registration of images, but particularly the MC-GAN algorithm can be applied for generation of images (250). The following non-limiting examples are given.

First example is generating fusion-image provided with all the input conditions (images from the first and second imaging modalities 110, 120, 210, 220), including optional human-controlled parameters. The optional human-controlled parameters could for instance map, define or otherwise represent whether the at least part of the tissue in the combined image should represent information coming from the first diagnostic image modality 110, 210. Second example is generating fusion-image provided only with diagnostic image information, i.e., the information coming from second diagnostic image modality 120, 220 alone, or in combination with the information from the diagnostic image with biopsy needle 290. In this case the trained MC-GAN can be used in image-to-image translation of the images.

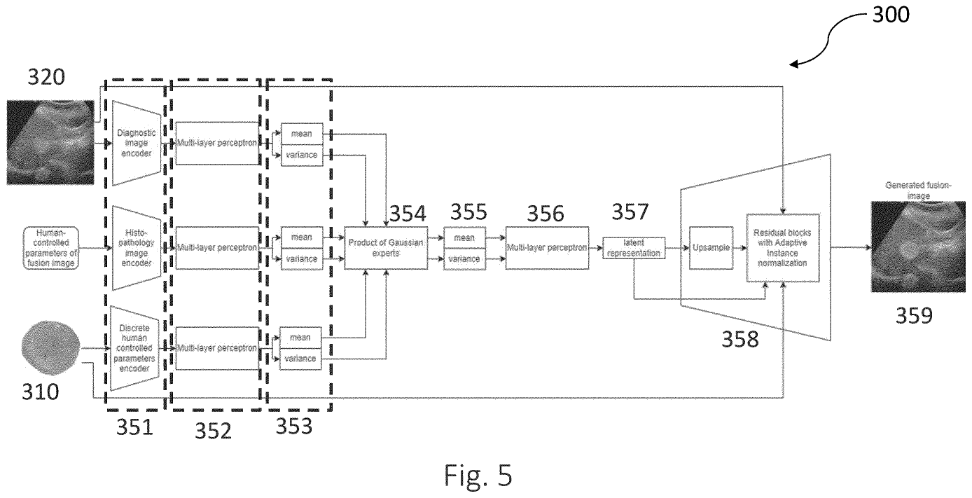

An example of such MC-GAN algorithm is provided with respect to FIG. 5. The example in relation to FIG. 5 will be described in respect to embodiments 300, but it is to be understood that the same principles apply to embodiments 100, 200. The MC-GAN described in FIG. 5 acquires data from the first pathological imaging modality 310 and second diagnostic imaging modality 320, wherein the input data can also contain human-controlled parameters of fusion of images. These inputs are called as “input channels” within the context of the present invention. For instance, additional discrete parametrized vector that define fusion-image appearance may be considered. Then the MC-GAN algorithm begins the analysis. The generator (not shown) may be trained to learn to create simulated/pseudo-data by e.g., incorporating feedback from the user-controlled parameters. For instance, the user may define specific weight, and the generator then learns the weights. Further, an encoder 351 encodes the data, wherein the decoder tries to reconstruct the data back using the internal representations and the learned weights. As a non-limiting example, a “product-of-expert” may be used, wherein the encoder is projecting input conditions into the joint conditional latent space. Other encoder types may be used, for instance Variational Autoencoders (VAE) or Vector Quantized VAE (VQ-VAE).

There might be different possibilities for encoding the images within the context of the present invention. For instance, when only a single condition (i.e., only the images from the second imaging modality) is passed through the encoder 351, latent space distribution associated with the encoder, is getting wider, which means the possible generated images by the image generation algorithm 350 can deviate from the actual images. However, since the latent space distribution is smaller, the combined, fused or otherwise synthesized images are generated much faster. The use case of such an image can be for screening purposes, where the precision is not critical, but speed is much more valued. On the other hand, for complex surgery, navigation for biopsy, and other complex clinical procedures, the system may consider information from all of the sources. However, when all the conditions (diagnostic image 310, 320, 390, as well as user input) are provided, the number of constraints increases, and the space of possible outputted fused images shrinks. In other words, the generated fused images will contain more details, and increase the size of the images. Depending on the goal and available storage data, different approaches may be used within the context of the present invention.

In step 352 a multi-layer perceptron is applied. In step 353 the mean and variance are calculated. Within the current invention, usage of mean and variance in step 353 is configured to describe data point(-s) in a latent space. A practical example is given: in each input channel, each encoder may “compresses” and project input condition (pathological images 310 and diagnostic images 320) to the feature space. Within the context of the present invention, this may mean that instead of representation of an image in a form of pixels in the latent space (e.g., 4096×4096 pixels), each condition may represent with a feature vector (e.g., 512×1 vector). Within the context of the present invention, projection and sampling may mean the standard meaning within the field. For instance, normal distribution around each point of the input data in the latent space may be calculated, and the mean μ(x)) and σ(x) may be the parameters of this distribution. These parameters may be different for each point.

In some embodiments, there are 3 pair of encoders that analyze the images from the input channels. In some other embodiments, there is 1 encoder, which analyzes the images from the input channels and human-input. However, other number of encoders is possible within the context of the present invention. The output of the encoder 351 with additional operations 352, 353 is called latent space distribution within the context of the present invention. In the step 354 and in an embodiment, Product of Experts, PoE (also called product of Gaussian Experts) is applied. Product of experts (PoE) is a machine learning technique, which models a probability distribution by combining the output from several distributions, i.e. in relation to diagnostic images 310, 320, and human input. Other parametric model types (e.g., mixture of Gaussians, generalized product of experts) might be used within the context of the present invention. The PoE in steps 354 may weight mean and variance of distributions in step 355 from the input parameters. The resulting mean and variance values may be passed to a multi-layer perceptron that in step 356 is used to create a vector-form latent representation 357 of the desired combined image. This latent representation 357 can be passed to the decoder 358 of the generator.

In some embodiments, the multi-layer perceptron 352, 356 (in a form of a fully connected neural network, convolutional neural network, attention based neural network) may be used as a mapping network for additionally performing the operation 340. The multi-layer perceptron (352, 356) allows to disentangle latent feature space which makes the conditioning of the whole neural network more controllable. Disentangling of the latent space leads that the input channels do not affect each other in latent space. Mean and variance in steps 352, 356 may describe the data points in the latent space. In some embodiments, the multi-layer perceptrons are the same. In some other embodiments, the multi-layer perceptrons 352, 356 are different. For instance, the multi-layer perceptron 356 may be more sophisticated as the latent space is more complicated and includes conditions of pathological 310 and diagnostic 320 images.

The decoder 358 comprises of a sequence of up-sampling layers and residual blocks with an adaptive instance normalization. The adaptive instance normalization layer accepts a scaled (i.e., processed) diagnostic image, a scaled (i.e., processed) pathology image, and a latent representation vector to form a desired output of the network by transferring key features of the input conditions. In some embodiments, decoder 358 can further include normalization algorithms, which normalize the expected result. One such example is Adaptive Instance Normalization, which is a normalization method that aligns the mean and variance of the content features with those of the style features. However, other normalization methods can be used within the context of the present invention. Finally, in step 359, the combined image is generated. However, it is to be understood that different variations of this algorithm are possible, depending on the goal at hand. For instance, the data from images 310 and 320 can be directly fed to the encoder 358 to have a very simplified version of the algorithm that can e.g., be used for screening purposes.

The MC-GAN of the current application may have the following structure. For instance, the encoders 351, 451 may contain convolutional layers with skip-connections. The multi-layer perceptrons 352, 356 may contain 4 fully connected layers. In some embodiments, the connected layers may further comprise a hidden dimension 4 times smaller than an input data dimensionality. The latent space may be represented with a vector space with e.g., 512 dimensions. Other dimensions may be possible within the context of the present application. The decoder 358 may contain residual blocks with convolutional layer(s). Each block may contain 4 convolutional layers with the number of filters 4 times smaller than the input channel size. The kernel size among the convolutional layers may be set to 3. The activation function layers applied in encoders, multi-layer perceptrons and decoders are “leaky ReLU” (rectified linear units) with slope 0.2. The skilled person would realize that other implementations may be possible.

Conventional MC-GAN's, like the one presented in Zhu Zhang, Jianxin Ma, Chang Zhou, Rui Men, Zhikang Li, Ming Ding, Jie Tang, Jingren Zhou, and Hongxia Yang. M6-UFC: Unifying multi-modal controls for conditional image synthesis or Huang, Xun, et al. “Multimodal conditional image synthesis with product-of-experts gans.” arXiv preprint arXiv: 2112.05130 (2021) is trained in generation output from a single modality, and designed to unify multi-modal controls. This framework is not designed to generate multi-modal images, nor it is designed generate diagnostic images. The current framework is trained to fuse input images. The input conditional images from two imaging modalities are preserved and present in the output image, while in the mentioned conventional MC-GAN's the input images are used to create new images with style from the input ones. The conventional MC-GAN's are unapplicable to diagnostic images since the images need to preserve a lot of information from the two modalities, and the conventional MC-GAN's are sketching-out the information. While the original GAN accepts textual description, segmentation mask, sketch, and style-reference image to produce image that never existed before, the MC-GAN of the present application fuses/generates existing images of two different diagnostic imaging modalities and further aligns the images to preserve the diagnostic information. Hence, since smaller number of conditions are used, the training of the model of the present application is simpler and more precise. Also, the number of input encoders is reduced. Thus, the overall complexity of the MC-GAN is decreased comparing to the original GAN.

It is to be understood that the described algorithm described in FIG. 5 is a non-limiting example, and other variations of this algorithm may be possible.

The algorithms described here may also be trained. A non-limiting example of training the MC-GAN algorithm is described in FIG. 6. For simplicity, the training of the model is described with respect to a separate embodiment 400, but it is to be understood that this model may apply also to embodiments 100 and 200.

The training of the model uses the input of the diagnostic images 420 from the first diagnostic imaging modality, both the diagnostic images 420 from the second diagnostic imaging modalities and images 410 from the first pathological imaging modalities. In some embodiments, also human input may be considered. Then, the MC-GAN algorithm 300 can be applied, like the one described in respect to FIG. 5. This generates the combined images 459 similar to the images in respect to embodiment 300, i.e., images 359. Optionally, the model 400 can use the combined images from previous steps 359 for training of images. An encoder 451 is applied at the next step. The encoder 351 is similar to the encoder 351 described in reference to the embodiment 300. Within the encoder 451, algorithms for optimization the encoder functioning can be used. For instance, Kullback-Leibler divergence can be calculated from a prior distribution to a conditional latent distribution, which aims to further optimizing the encoder. In the next step 451a, the loss values are calculated. The loss values may feed back to the encoder 451 and/or the MC-GAN algorithm 300 for training of images. The loss values may be of 3 principal types: image contrastive loss, conditional contrastive loss, and adversarial loss. Within the present invention, when the loss values calculation is being referred to, one or more of these loss types are referred to. Further a short description of the different loss types is presented. Image contrastive loss maximizes the similarity between real and random fake image synthesized based on the corresponding conditional inputs. Conditional contrastive loss aims to better align synthesized images with the corresponding conditions. Adversarial loss is measuring how realistic is generated image from the discriminator point of view. When a sufficiently good result is achieved, the GAN network stops the training and generates the final result. The training stops once the generator's and discriminator's loss curves achieve plateau, and the metrics calculated on the validation set are not improving anymore with each subsequent epoch of training. The generated final result includes combined image 459.

The training may be based on the following non-limiting implementation example. In a non-limiting example, the current method is trained based on the Adam optimization technique. The skilled person would know that Adam is an algorithm for first-order gradient-based optimization of stochastic objective functions, based on adaptive estimates of lower-order moments. Other algorithms may be possible within the context of the present application: AdaDelta, Adagrad, Nesterov accelerated gradient, and others.

Adam optimization technique with parameters β1=0 and β2=0.99 may be used to train the MC-GAN of the present application. The weights of the generator at the end of training are defined by an exponential moving average of its weights during training. Dropout of input data modalities with 50% rate is applied during training to make the MC-GAN more stable to the missing input conditions. Learning rate scheduling is applied with weights' rebalancing with a decay factor equal to 0.99 (weight decay method). Early stopping in a form of metrics and losses monitoring is applied to prevent MC-GAN from overfitting. The minimum number of training iterations is set to 1000 epochs with minimal batch size equal to 4 images. Other variations within the context of the present application may be possible.

The methods 100, 200 and the associated MC-GAN models 300, 400 may work in two or more modes. The first mode may be the image mapping mode that includes all the components shown in FIG. 1. The second mode may be an image-to-image translation mode where the trained MC-GAN network may be used. In other words, the algorithms 100, 200 may be AI (Artificial Intelligence) and non-AI based.

The image mapping mode may be of a) Asynchronous image fusion based on image mapping; b) Synchronous image fusion based on image-to-image translation. Other modes are possible.

The first mode, i.e., the image mapping mode, may be asynchronous since it may require acquiring diagnostic images 120, 220 first, then conducting image-guided biopsy 110a, 220a with biopsy region extraction 296. Furthermore, performing the image registration 230a can be done with sequential fusion-image synthesis. Providing two conditions (diagnostic images 120, 220 and pathological images 110, 210) may impose additional restrictions and make the synthesis task less ill-posed. As a result, detailed information-enriched fusion-image is generated. The outcome can be used by physician as a support for medical decision making. This mode may be deployed on the diagnostic device (e.g., US) or in a separate workstation.

The second mode allows synchronous image-to-image translation in real time. The trained model may be deployed directly on a diagnostic device, which is capturing the diagnostic images 120, 220. These images are passed through the generator which synthesizes the pathological overlay on top of the diagnostic images 120, 220. This mode may be faster than the first described mode. However, the speed of operation and the simplicity of use allow this mode to be used for high-throughput screening, providing physicians with additional information about probable morphology of tissues in a diagnostic image in real time.

As would be apparent for the skilled person, the application of GAN's or MC-GAN's to generation of a synthesized image is one of the insights of the present application. Even though the GAN itself is known, the application of the GAN/MC-GAN to generation of images from two imaging modalities is not apparent for the skilled person. The known methods, such as multi-modality image simulation used for radiation therapy planning, is using the imaging modalities from the same modality type, e.g., CT and MR.

In some examples, a system for storing the data is described. The system for storing the data could store both diagnostic images, pathological images, or combined images. In some examples, the system can perform compression of images. Any type of compression algorithms known in relation to diagnostic or pathological images can be used. In some examples, the generated combined image may be transmitted to a Tumor Board system, or displayed to a Radiological/Pathological workstation, or present in real-time.

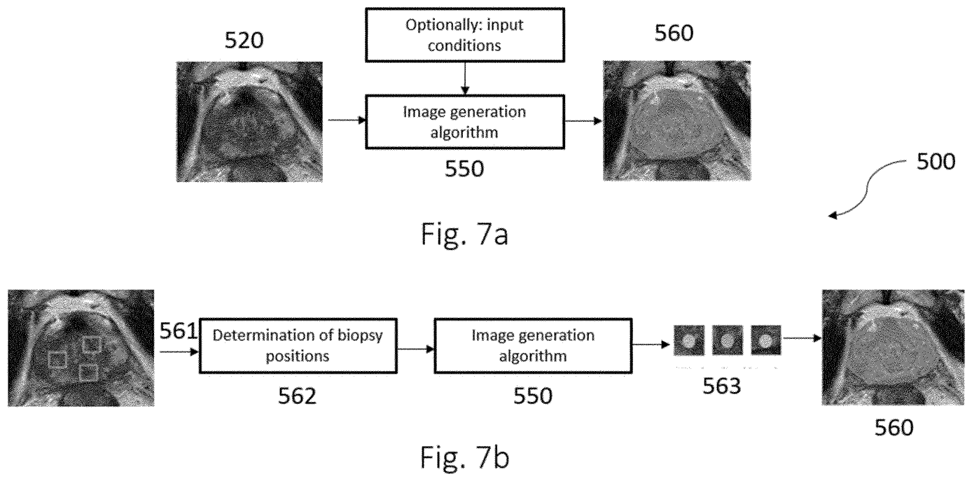

FIG. 7 describes another embodiment of the present application. The embodiment is purely for better understanding of the previously described embodiments, and is not limiting in any way possible. The embodiment refers with respect to MR image data and combination of information with respect to MR image data.

According to the embodiment of FIG. 7, an approach for generation of the cellular information is described. Exemplary, the approach is described by using a generative AI model algorithm type, but in principle other types of machine learning algorithms could be used.

According to the first embodiment of FIG. 7 (FIG. 7a), radiological image data (520) and pathological image data as obtained from at least one (known) biopsy location as an input. The image generation algorithm (550) is applied to generate and visualize a combined image (560). Optionally, additional input conditions, such as biopsy information, cellular information, image information at biopsy positions, and/or biopsy positions in X, Y, Z coordinates can be applied and fed to the image generation algorithm for improvement of results. Preferably, the image generation algorithm is a machine learning algorithm of generative AI type.

FIG. 7b describes an embodiment, wherein the images patches are extracted (561), biopsy locations are determined (562), based on the determined biopsy locations, an image generation algorithm (550), preferably a machine learning algorithm of generative AI type, is applied to extract, translate or otherwise determine (563) cellular information/cellular markers for patch centers, wherein based on this information, a combined image is visualized (560). Preferably, the visualized image includes image patch information and/or information about cellular information/cellular markers for patch centers.

In an example, the set of radiological images comprises of one MR image and a set of (live) ultrasound images. The way the biopsy locations are known in this example is by markers in at least one of the ultrasound images or by using e.g., an electromagnetic tracker attached to the biopsy needle. Based on the acquired information, a registration module registers the biopsy locations and the biopsy histological information to at least one target radiological image from the radiological image data out of the at least one input radiological images. After that the biopsy information is attached to a set of locations in the radiological image. Based on this, a biomarker extraction module determines cellular information from the pathological information data (e.g., obtained from biopsies and scanned with a pathology scanner) as an input and extracts a set of cellular information (e.g., cellular biomarkers) that characterize the image. Examples for such biomarkers are mentioned in earlier sections of this document and include gleason scoring or cell growth markers. Optionally, a refinement module based on a generative AI model, which could be a pre-trained foundational model, obtains the registered information as an input, and refines the generative AI model based on this input data to tailor it to already available support information for the current patient and generate the information on the screen, like score information. This information is then displayed on the display or otherwise communicated to the user of the system.

In some embodiments, the generative AI model obtains radiological image data as an input and outputs predicted pathological information data as output. In some cases, the data is predicted based on a pointwise algorithm for at least some of the locations for at least some of the images in the radiological image data dataset. The information at the support locations where the biopsy was taken is preserved as it is already known for this patient and for all other locations it is extrapolated based on these support locations. Optionally, the generative AI model also outputs a map that indicates the confidence of the extrapolation information at a specific location.

In some embodiments, the generative AI model is refined for each new patient based on the information from the previous patient. Base model for this refinement is always the same model and not the refined model from the last patient as this may be too tailored to the last patient an a less optimal starting point for the next patient.

The registration of images by the registration module can have different implementations dependent on the embodiment. For instance, for the embodiment of combining live US images with MR images, the registration module could be based by tracking different information points in the same space. For instance, by tracking electromagnetic markers that are attached to a biopsy needle as well as the ultrasound probe, so that by tracking all markers their relative position in the same coordinate system is known as well as their relative position with respect to other known landmarks such as the needle tip or US transducer plane. For the case of diagnostic (e.g., MR) to pathological image registration, other registration approaches could be used.

In some embodiments, an objective function could be used for improvement of the registration algorithm with respect to a parameterized image-to-image transformation. The objective function can be a similarity function that compares both images after iteratively applying a spatial transformation (linear or non-linear deformation) which yields proposals for new transformation parameters at each iteration that are predicted to result in an even better similarity score. A pre-processing step for both images to transfer them into another representation could be introduced. This has an advantage of that is better suited to estimate the spatial transformation, e.g., when registering different modalities it may be beneficial to first semantically segment both modalities delineating structures that appear in both images.

In some embodiments, an iterative registration process is used. In this embodiment, the images are called registered when an optimal point in the objective function is reached and both images are transformed into the same coordinate space. In an example the biopsy needle is registered to the live ultrasound images (used to guide the biopsy) via electromagnetic tracking. The US images are registered to an MR image by image-to-image registration and not necessarily in the target anatomy depending on which overlapping anatomical regions are best suited for registering US and MR images based on structures visible in both modalities. As a result the MR image with N biopsy locations in the volume for which biomarker vectors have been measured is input to the subsequent generative model.

The biomarker extraction module as described previously could be a signal processing element (e.g., texture filtering circuit and algorithm) or a learned model. In some embodiments, a set containing at least one cellular biomarker (e.g., Gleason score, cell growth, lesion shape, cell diffusion) may provide the target information that shall be extracted from the histological image and result in a biomarker vector. This biomarker vector may be extended by its spatial coordinates in the radiological image coordinate space as well as the image intensities of the radiological image at these locations

Generative AI model at least in some embodiments may refer to a model that takes a radiological image as an input and generates a biomarker vector at a certain location in the radiological image of the radiological image data. The model may also take a spatial mask that restricts the number of locations for which the biomarker vector shall be predicted to a target region (e.g., the prostate) only. The model may be trained from random initializations or use a foundation model.

At least in some embodiments, the model may process the whole image at once to predict the biomarker information at each location at once or decompose the image into smaller patches and predict a biomarker vector for the center of each patch. As shown in FIG. 2, the model is trained accordingly. Examples for appropriate generative models use a convolutional or transformer-based architectures. Optionally, before the deployment in the clinical diagnosis the model is trained on a (large) number of (radiological image(s), biomarker vector(s)) pairs, such that it learns to provide predictions that generalize well across a cohort of patients, but that may not be specifically tailored to each individual patient. The latter is taken care of by the refinement module when the invention is deployed for a specific patient. Optionally, the model outputs a confidence score for each prediction at each location. Possible implementations include test-time drop-out/augmentation (epistemic uncertainty) or a predicted confidence map as a parallel output (aleatoric uncertainty). In case of patch-wise prediction, additional out-of-distribution models can be used to predict a prognostic confidence of the generative model. Optionally, the confidence map may be used to plan additional biopsies or to discard planned biopsies.

At some embodiments, the refinement module performs actions similar to the training of model, but at the time of the deployment of the intervention to tailor the model to the patient-specific information obtained from biopsies extracted as part of the diagnostic workflow, i.e. obtaining “support information” as it does not need to be predicted and is already known. The refinement model is motivated by the idea that locations surrounding the support information are similar to the known vectors, such that the model should reproduce the support information at their respective locations and should extrapolate on the unknown locations based on the support information from this particular patient. The refinement process may depend on the model architecture. In some non-limiting embodiments, the refinement process can be carried out by 2 ways: (1) by changing the model parameters based on an optimization objective similar to the training to (over) fit the model to the patient-specific biopsy information (patient specific fitting), and/or (2) by computing parts of the model parameters based on the patient-specific biopsy information in a deterministic manner (patient-specific conditioning). Further details with reference to biomarker vectors will be provided with an understanding that this could be used for other cellular information.

In the case of patient-specific fitting, the biomarker vectors may be used as ground truth target output for the model together with input patches cut from the radiological image at the locations of the corresponding biopsies. Based on an objective function that can be the training loss function, the model parameters are optimized in such a way that the model optimally predicts the individual biomarker vectors from the corresponding support patches. Optionally, the model may take for each patch the patch position in the full volume or an anatomical space as an additional input. The refined model may be applied to all patches the whole image was decomposed into. This is to be understood to be an exemplary embodiment, which is not limiting in any way possible.

In the case of patient-specific conditioning, the biomarker vectors may be given as a condition that modify the way the processing steps of the model operate, the model may not be retrained based on some objective function (i.e., optimization such as gradient descent can be omitted), but some adaptable parameters of the model are computed based on the biomarker vectors may be given as conditions instead. In this embodiment, the model can operate on the whole radiological image while attending to conditional information that is provided for the support locations of the biopsy samples.

An example of such an architecture is a latent diffusion model or another transformer-based architecture.

The described disclosure may be provided as a computer program, or software that may include a computer-readable storage medium having stored thereon instructions, which may be used to program a computer system (or other electronic devices) to perform a process according to the present disclosure. A computer-readable storage medium includes any mechanism for storing information in a form (e.g., software, processing application) readable by a computer. In various implementations, the processor may be associated with one or more storage media such as volatile and non-volatile computer memory. Such as the computer-readable storage medium may include, but is not limited to, optical storage medium (e.g., CD-ROM), magneto-optical storage medium, read only memory (ROM), random access memory (RAM), erasable programmable memory (e.g., EPROM and EEPROM), flash memory, or other types of medium suitable for storing electronic instructions. RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor.

Variations to the disclosed embodiments can be understood and effected by those of ordinary skilled in the art in practicing the claimed invention, from a study of the drawings, the disclosure and the appended claims. In the claims, the word “comprising” does not exclude other elements or steps, and the indefinite article “a” or “an” does not exclude a plurality.

It is understood that one or more of the embodiments of the invention may be combined as long as the combined embodiments are not mutually exclusive. As will be appreciated by one skilled in the art, aspects of the present invention may be embodied as an apparatus, method or computer program product. Accordingly, aspects of the present invention may take the form of an entirely hardware embodiment, an entirely software embodiment (including firmware, resident software, microcode, etc.) or an embodiment combining software and hardware aspects that may all generally be referred to herein as a “device”, “module” or “system.” Furthermore, aspects of the present invention may take the form of a computer program product embodied in one or more computer readable medium(s) having computer executable code embodied thereon.

Functions implemented by a processor may be implemented by a single processor or by multiple separate processing units which may together be considered to constitute a “processor”. Such processing units may in some cases be remote from each other and communicate with each other in a wired or wireless manner. A processor may include a software executing device and/or dedicated hardware, such as an application-specific integrated circuit (ASIC) and/or a field-programmable gate array (FPGA).

Measures recited in mutually different dependent claims may be advantageously combined.

A computer program may be stored/distributed on a suitable medium, such as an optical storage medium or a solid-state medium supplied together with or as part of other hardware, but may also be distributed in other forms, such as via the Internet or other wired or wireless telecommunication systems. A computer-readable storage medium can be used.

As will be appreciated by one skilled in the art, aspects of the present invention may be embodied as an apparatus, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer executable code embodied thereon. Any combination of one or more computer readable medium(s) may be utilized. The computer readable medium may be a computer readable signal medium or a computer readable storage medium. A ‘computer-readable storage medium’ as used herein encompasses any tangible storage medium which may store instructions which are executable by a processor or computational system of a computing device. The computer-readable storage medium may be referred to as a computer-readable non-transitory storage medium. The computer-readable storage medium may also be referred to as a tangible computer readable medium. A computer readable signal medium may include a propagated data signal with computer executable code embodied therein, for example, in baseband or as part of a carrier wave.

‘Computer memory’ or ‘memory’ is an example of a computer-readable storage medium. Computer memory is any memory which is directly accessible to a computational system. ‘Computer storage’ or ‘storage’ is a further example of a computer-readable storage medium. Computer storage is any non-volatile computer-readable storage medium. In some embodiments computer storage may also be computer memory or vice versa.

Machine executable instructions or computer executable code may comprise instructions or a program which causes a processor or other computational system to perform an aspect of the present invention. Computer executable code for carrying out operations for aspects of the present invention may be written in any combination of one or more programming languages, including an object-oriented programming language such as Java, Smalltalk, C++ or the like and conventional procedural programming languages, such as the “C” programming language or similar programming languages and compiled into machine executable instructions. In some instances, the computer executable code may be in the form of a high-level language or in a pre-compiled form and be used in conjunction with an interpreter which generates the machine executable instructions on the fly. In other instances, the machine executable instructions or computer executable code may be in the form of programming for programmable logic gate arrays.

Aspects of the present invention are described with reference to flowchart illustrations and/or block diagrams of methods, apparatus (systems) and computer program products according to embodiments of the invention. It is understood that each block or a portion of the blocks of the flowchart, illustrations, and/or block diagrams, can be implemented by computer program instructions in form of computer executable code when applicable. It is further understood that, when not mutually exclusive, combinations of blocks in different flowcharts, illustrations, and/or block diagrams may be combined. These computer program instructions may be provided to a computational system of a general-purpose computer, special purpose computer, or other programmable data processing apparatus to produce a machine, such that the instructions, which execute via the computational system of the computer or other programmable data processing apparatus, create means for implementing the functions/acts specified in the flowchart and/or block diagram block or blocks.

The computer executable code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

If the term “adapted to” is used in the claims or description, it is noted the term “adapted to” is intended to be equivalent to the term “configured to”. If the term “arrangement” is used in the claims or description, it is noted the term “arrangement” is intended to be equivalent to the term “system”, and vice versa.

Procedures like acquiring image data from the first pathology imaging modality, acquiring image data from the second imaging modality, registering the images, mapping the images, applying an image generation device, visualizing the images, et cetera, performed by one or several units or devices can be performed by any other number of units or devices. These procedures can be implemented as program code means of a computer program and/or as dedicated hardware.

Any reference signs in the claims should not be construed as limiting the scope.

### REFERENCE NUMERALS

110, 220, 320, 420: pathological image data, pathological images, first pathological imaging modality
120, 220, 320, 420: diagnostic image data, diagnostic images, second diagnostic imaging modality
130, 230, 230a: registration of the pathological image data to the diagnostic image data
140, 240: mapping of the pathological image data to the diagnostic image data
150, 250: applying an image generation algorithm
160, 260, 260a: visualizing the at least one combined image.
110a, 220a: image-guided biopsy
110b, 220a: Obtain and process the samples acquired from image-guided biopsy
120a, 220a: performing a diagnostic imaging study
170, 270: a biopsy localization processor and corresponding method of biopsy localization
180, 280: second diagnostic imaging modality with the biopsy needle
190, 290: diagnostic image with a biopsy needle
291: performing the down-sampling of the layers
292: perform global feature extraction
293: fusion of images
294: application of classifier and decoder
295: applying a segmentation mask
296: a biopsy extraction
297: biopsy region is acquired
351, 451: encoder
353: calculation of mean and variance
352, 356: multi-layer perceptron
354: Product of experts (PoE)
355: weighting mean and variance of distributions
357: vector-form latent representation
358: decoder
359, 459: generation of combined image
561: extraction of image patches
562: determination of biopsy positions
563: cellular information/cellular markers for patch centers

Abbreviations:

| | |
|---|---|
| CNN: | Convolutional Neural Network |
| GAN: | Generative Adversarial Network |
| MC-GAN: | multi-conditional generative neural network (MC-GAN) |
| DICOM: | Digital Imaging and Communications in Medicine |
| HFUS: | High Frequency Ultrasound |
| HIFU: | High Intensity Focused Ultrasound |
| US: | Ultrasound |
| CT: | Computed Tomography |
| MR: | Magnetic Resonance |
| MRI: | Magnetic Resonance Imaging |
| ML: | Machine Learning |
| NN: | Neural Network |
| I2I : | image-to-image |
| PET: | Positron Emission Tomography |
| SPECT: | Single Photon Emission Computed Tomography |
| H&E: | Hematoxylin and Eosin |
| RVSS: | Register Virtual Stack Slices |

1. A computer-implemented image generation method for generating combined diagnostic imaging and pathology images, the method comprising:
acquiring, with a first imaging modality, pathological image data of biopsied regions of a subject;
acquiring, with a second imaging modality, diagnostic image data of the subject;
mapping the pathological image data to the diagnostic image data; and
applying an image generation algorithm in order to generate a combined diagnostic and pathology image based on the mapping of the data, wherein the image generation algorithm is a machine learning algorithm refined based on mapping information, and wherein the image generation algorithm is further configured for determining cellular information from the pathological image data of biopsied regions of a subject, and extrapolating this information to non-biopsied regions for determining cellular information of non-biopsied regions.

2. The method according to claim 1, wherein the method includes generating a confidence map indicating a confidence of determined cellular information of non-biopsied regions.

3. The method of claim 1, wherein a biomarker extraction module is used for calculating and/or extracting cellular information from the pathological image data of biopsied regions and extrapolating this information to non-biopsied regions.

4. The method of claim 3, wherein based on the mapping, the biomarker extraction module predicts histological information for the locations in the radiological image data other than locations corresponding to the pathological image data of biopsied regions.

5. The method of claim 1, wherein the image generation algorithm is refined based on the cellular information as an optimization target.

6. The method of claim 1, wherein the cellular information comprises any one of: Gleason score, cell growth progression, cell growth markers, lesion shape, cell diffusion, and wherein based on the determined cellular information, a biomarker vector is calculated.

7. The method of claim 6, wherein the biomarker vectors are used as input for the image generation algorithm, wherein the image generation algorithm is configured for determining N biopsy locations in Region of Interest, ROIs, for which biomarker vectors have been measured.

8. The method of claim 1, wherein the diagnostic image data is a Magnetic Resonance, MR, image data, and wherein MR intensities are calculated based on the MR image data, the MR intensities further being used to calculate cellular information.

9. The method of claim 1, wherein a refinement module is used for adapting the model parameters of the image generation algorithm based on an optimization objective, and/or wherein the refinement module is configured to calculate model parameters based on patient-specific biopsy information.

10. The method of claim 1, wherein the image generation algorithm is further configured to be refined based on the cellular information from a patient, and wherein the image generation algorithm is further optimized to analyze the data of the patient before the algorithm extrapolates the information to non-biopsied regions of that patient.

11. The method of claim 1, wherein based on the pathological data, cellular biomarkers are extracted, and wherein the cellular markers are fed into the machine learning algorithm as an optimization target.

12. The method of claim 1, wherein an extraction module is used for the extraction of cellular information, and wherein the extracted cellular information is fed to the machine learning algorithm, wherein the extraction module has fixed parameters used for extraction of cellular information.

13. The method of claim 1, the method comprising generating at least one whole-organ histology image comprising of diagnostic image data, and pathological image data of biopsied and extrapolated non-biopsied regions.

14. The method of claim 1, wherein image intensities are calculated for the diagnostic image data, wherein image intensities are used for generating image patches based on the diagnostic image data.

15. The method of claim 14, wherein a patch-wise prediction for image patches of non-biopsied regions is determined, and a confidence score for generated image patches is calculated.

16. The method according to claim 1, wherein the image generation algorithm is configured to learn and synthesize the pathological representations covering the whole or part of tissue in the diagnostic image data.

17. The method of claim 1, wherein diagnostic image data is MR image data, and wherein the MR image data is registered with live US image data from an Ultrasound machine, wherein at least one of the following model parameters are determined: MR image intensities, cellular markers, biopsy locations in the MR image, and wherein based on these parameters, a confidence registration map is determined, the confidence registration map being reflective of registration certainty of MR image data with live US image data.

18. The method of training an image generation algorithm to be deployed in claim 1, wherein the algorithm is trained based on the diagnostic image data, or based on both the diagnostic image data and the pathologic image data, and wherein the training is done based on a Generative Adversarial Network (GAN) algorithm, a diffusion model algorithm, a convolutional neural network and/or transformer-based neural network.

19. A system for generating combined diagnostic imaging and pathology images, the system comprising of:
first imaging modality configured to generate pathological image data of biopsied regions of a subject;
second imaging modality configured to generate diagnostic image data of the subject;
a processor;
the system further configured to:
acquire pathological image data of biopsied regions of a subject from the first imaging modality;
acquire diagnostic image data from the second imaging modality;
map the pathological image data to the diagnostic image data; and
the processor configured to execute an image generation algorithm in order to generate a combined diagnostic and pathology image based on the mapping of the data, wherein the image generation algorithm is a machine learning algorithm trained based on mapping information, and wherein the image generation algorithm is further configured for determining cellular information from the pathological image data of biopsied regions of a subject, and extrapolating this information to non-biopsied regions for determining cellular information of non-biopsied regions.

20. A computer program configured to enable a processor to carry out the method of claim 1.
